(19) United States
(12) Patent Application Publication
(10) Pub. No.: US 2021/0329989 A1
(43) Pub. Date: Oct. 28, 2021

(54) UV Sterilizer Face Mask Optimized with a Flowmeter, Electrostatic Trap, and Ultrasonics

(71) Applicant: Javad Rezanezhad Gatabi, San Marcos, TX (US)

(72) Inventor: Javad Rezanezhad Gatabi, San Marcos, TX (US)

(21) Appl. No.: 17/241,109

(22) Filed: Apr. 27, 2021

Related U.S. Application Data
(60) Provisional application No. 63/101,380, filed on Apr. 27, 2020.

(51) Int. Cl. A41D 13/11; A61L 9/20; A61L 9/22

(52) U.S. Cl. CPC A41D 13/11; A61L 9/20; A61L 2202/122; A61L 2202/14; A61L 9/22

(57) ABSTRACT

This invention introduces a compact, power efficient sterilizer face mask combining multiple technologies to effectively stop the spread of airborne microbial diseases, bacteria, and viruses. The geometrical design of the system allows for a longer air-flow pathway inside the optical box to increase the exposure time. An electrostatic or ionizing structure, traps the bacteria and viruses and slows down their movement inside the mask, significantly increasing the light exposure time. In addition, the air can flow through thermal (heating or cooling) engines and sonic or ultrasonic exposure engines for increasing the whole system's effectiveness. The UV exposure unit, the electrostatic unit, the thermal unit, and the sonic/ultrasonic unit can be smartly activated or deactivated using an air flowmeter that significantly reduces the power consumption of the system and its generated heat. The flowmeter can also selectively control the power of each unit depends on the flow rate or flow direction of the air inside the system.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The proper wavelengths and dosage (a product of intensity and exposure time) of UV light can be effective at killing bacteria and viruses by destroying their molecular bonds holding their DNA. The idea of integrating UV light sources with face masks results in an attractive instrument to kill or inactivate bacteria and viruses while breathing. Achieving an acceptable result, requires the use of high power UV light sources and increasing the exposure time by sterilizing in a large volume container. Hence, the integration of UV light sources with face masks in a compact instrument is challenging due to the power consumption, generated heat, and the effectiveness of the system.

The breathing (respiration) rate for a normal adult varies around 12-20 breaths per minute at rest. The Tidal volume (TV), the volume of displaced air between the normal inhalation and exhalation, for a young human is around 0.5 Liter per inhalation. Combining these two values, the average breathing flow rate for a normal young human is in the range of tens of Liter per minute (L/min). In addition, the peak flow rate can be in the range of several hundreds of L/min. In order to kill above 90% of most bacteria and viruses, the UVC dosages of several thousands μW·s/cm² is required. So, an effective UV-based face mask with a practical dimensions (with tens of cubic centimeter or hundreds of cubic centimeter volume) requires UV light sources with several or tens of mW/cm² irradiance at the effective distance for real-time sterilizing the air while breathing. Such a high power light source, specially at UVC spectra where the most effective wavelength falls, requires significant electrical power source. In addition, due to the low efficiency light sources, the generated heat will be uncomfortable at proximity of the human face.

### BRIEF SUMMARY OF THE INVENTION

This invention introduces a compact, power efficient sterilizer face mask combining multiple technologies to effectively stop the spread of airborne microbial diseases, bacteria, and viruses. The geometrical design of the system allows for a longer air-flow pathway inside the optical box to increase the exposure time. An electrostatic or ionizing structure, traps the bacteria and viruses and slows down their movement inside the mask, significantly increasing the light exposure time. In addition, the air can flow through thermal (heating or cooling) engines and sonic or ultrasonic exposure engines for increasing the whole system's effectiveness. The UV exposure unit, the electrostatic unit, the thermal unit, and the sonic/ultrasonic unit can be smartly activated or deactivated using an air flowmeter that significantly reduces the power consumption of the system and its generated heat. The flowmeter can also selectively control the power of each unit depends on the flow rate or flow direction of the air inside the system.

### BRIEF DESCRIPTION OF THE SEVERAL VIEWS OF THE DRAWING

FIG. 1A illustrates an example embodiment of the sterilizer mask comprising an optional face mask and a sterilizer chamber. FIG. 1B illustrates an example embodiment of the sterilizer mask comprising an optional face mask and two sterilizer chambers and one one-way valve.

FIG. 2 illustrates an example embodiment of the sterilizer chamber comprising two air-exchange windows, at least one light source, and optional internal walls to increase the length of the air flow path.

FIG. 3A illustrates an example embodiment of the sterilizer chamber improved by employing a pre-charged plate to attract and trap the particles on the air to slow down their movement.

FIG. 3B illustrates an example embodiment of the sterilizer chamber improved by employing a plate charged with an electric charge generator to attract and trap the particles on the air to slow down their movement.

FIG. 4A illustrates an example embodiment of the sterilizer chamber improved by employing a pair of pre-charged plates to attract and trap the particles on the air or repel them to slow down their movement. FIG. 4B illustrates an example embodiment of the sterilizer chamber improved by employing a pair of plates charged with an electric charge generator to attract and trap the particles on the air or repel them to slow down their movement.

FIG. 5 illustrates an example embodiment of the sterilizer chamber improved by employing one pre-charged single-plate, one pair of pre-charged plates, one single-plate charged with an electrical charge generator, and one pair of plates charged with an electrical charge generator. The system is further improved employing an air ionizer device.

FIG. 6 illustrates an example embodiment of the sterilizer chamber improved by employing a controller unit that can turn on and off the bacteria killing/deactivating units and the electrical charge generator units and or adjust/control their intensity, energy, power, or effectiveness.

FIG. 7 illustrates an example embodiment of the sterilizer chamber improved by employing a flowmeter to detect the existence of the air flow and or to measure the air flow rate and or to measure the speed of the air and or to measure the air flow direction. The data measured by flowmeter is used by the controller unit to control different units via control signal lines.

### DETAILED DESCRIPTION OF THE INVENTION

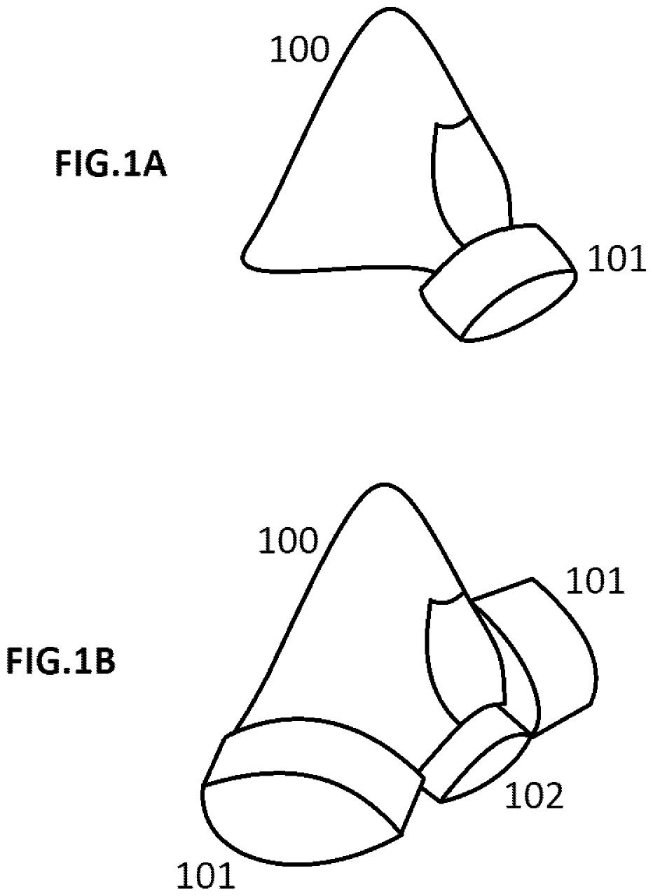

This sterilizer face mask system (named sterilizer mask) can be implemented as an entire mask system or as an instrument attachable to the current standard masks or as an instrument attachable to any custom designed mask. In its simplest form, as shown in FIG. 1A, this sterilizer mask is comprised of an optional face mask 100 and a sterilizer chamber 101. Where the sterilizer chamber 101 can be made in many shapes, with any dimensions and volume. It must be noted that the sterilizer mask system can also be implemented using more than one sterilizer chamber 101. An example is illustrated in FIG. 1B wherein two sterilizer chamber 101 are employed. The sterilizer face mask system can also be equipped with one or more one-way valve 102 that each one can be either an exhalation valve or an inhalation valve.

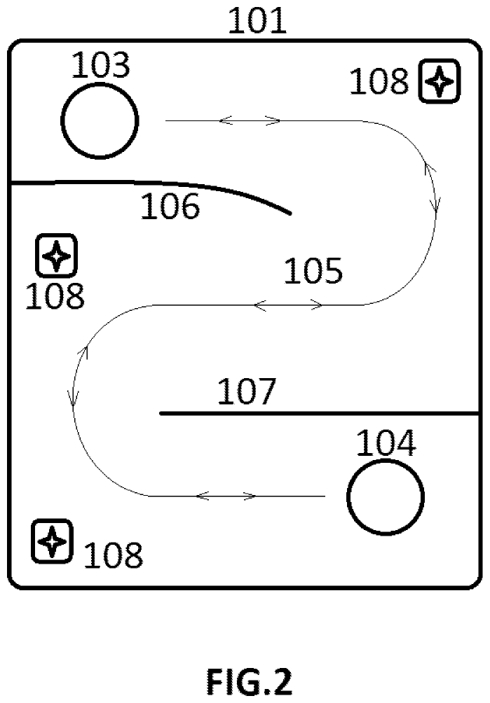

As shown in FIG. 2, The sterilizer chamber 101 is an air-tight chamber with at least two air-exchange windows wherein the outside window 103 exchanges the air with the outside ambient and the inside window 104 exchanges the air with the inside of the face mask 100. The outside window 103 and the inside window 104 can have any geometrical shape and may be placed with any orientation and anywhere on sterilizer chamber 101 such as on top plate, bottom plate, or on its side walls. While the user breathing, the air makes a flow path 105 inside the sterilizer chamber 101. If no one-way valve 102 is used in the sterilizer face mask system, on inhalation phase of breathing, the direction of flow path 105 is from the outside window 103 to the inside window 104 and on exhalation phase of breathing, the direction of flow path 105 is from the inside window 104 to the outside window 103. In the case of using one-way valve 102, the flow path 105 can be only on one direction depends on the direction of one-way valve 102.

If the sterilizer face mask system is intended to protect the user from the microbial diseases, viruses and bacteria on the ambient around the user, the one-way valve 102 should be installed in a way that the air can only flow from window 103 to window 104. If the sterilizer face mask system is intended to prevent the spread of microbial diseases, viruses and bacteria from the user, the one-way valve 102 should be installed in a way that the air can only flow from window 104 to window 103. If the sterilizer face mask system is intended to protect the user from the microbial diseases, viruses and bacteria on the ambient around the user and also to prevent the spread of microbial diseases, viruses and bacteria from the user, there is no need for the one-way valve 102.

It must be noted that the one-way valve 102 can be placed anywhere in the flow path 105 or anywhere outside the windows 103 or 104. It is also practical to have variations of multiple one-way valve 102 with multiple sterilizer chamber 101. For example, a sterilizer face mask system equipped with two sterilizer chamber 101 and two one-way valve 102 can be designed in a way that during the inhalation phase of breathing, the air flows inside the first combined sterilizer chamber 101 and one-way valve 102 and during the exhalation phase of breathing, the air flows inside the second combined sterilizer chamber 101 and one-way valve 102. The air flow can also be artificially made by means of a fan or an air pump.

It is also practical to increase the length of the flow path 105 using one or more than one internal wall(s) or obstacle(s) such as wall 106 or wall 107. The wall 106 and wall 107 may have any shape or orientation. The sterilizer chamber 101 can also be made in a geometrical shape to increase the length of flow path 105. One or more than one light source(s) 108 are placed along the air flow path 105. The volume of the air inside the sterilizer chamber 101 has a critical rule on the effectiveness of the sterilizer face mask system. In the preferred embodiment of this invention, the summation of the volumes of all synchronized sterilizer chamber 101, is equal or larger than the Tidal Volume of the user. The term synchronized refers to all sterilizer chamber 101 that make an air flow path 105 at the same time while the user breathing. Making the total synchronized sterilizer chamber 101 volume above the Tidal Volume of the user, practically makes one breathing-length delay on the air flow. It provides a longer light exposure time inside the sterilizer chamber 101 and significantly increases the effectiveness of the sterilizer face mask system. It is a bold novelty of the presented invention.

It must be noted that while Making the total synchronized sterilizer chamber 101 volume above the Tidal Volume of the user significantly improves the effectiveness of the system, selecting a lower volume can also be practical and still effective. For example, the total sterilizer chamber 101 volume can be selected 10% of the user tidal volume, or any value between 10% to 100%.

The light source 108 may have any wavelength on visible or outside of the visible spectra. However, the light sources at the UV-A, UV-B, or UV-C spectra are preferred because of their effectiveness on killing the microbial diseases, bacteria and viruses. The system may also be made with several different light sources with different wavelengths. The system can also include one or multiple light detectors to measure or monitor the intensity or the optical energy or the optical power of the light sources. The light detector can be employed for measuring the light source degradation and also detecting any blockage by dirt, dusts, and vapors. The variation of the light detector signal can also be used to count the particles in the air or measuring their size.

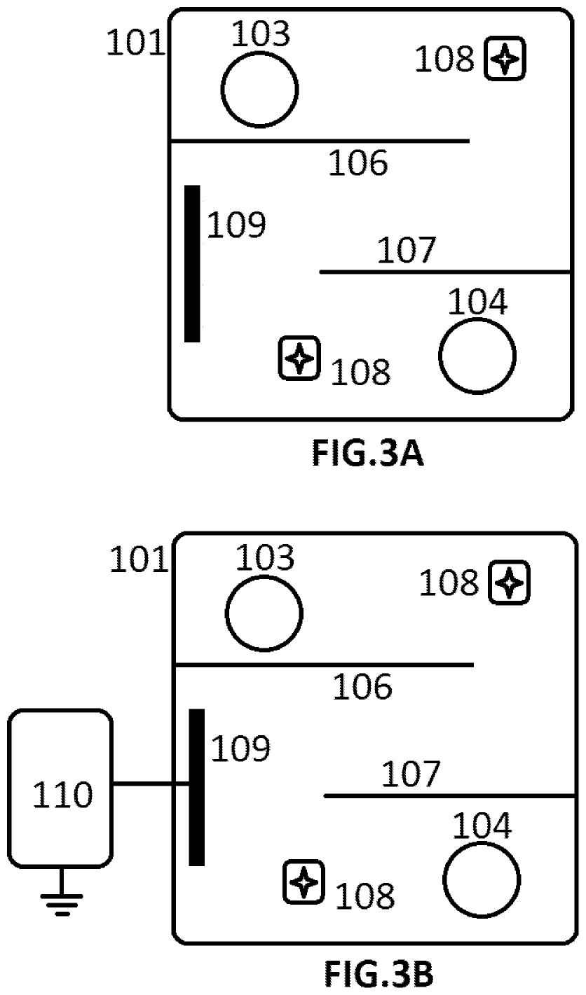

Another significant novelty of this invention is the use of an electrostatic trap slowing down the movement of microbial diseases, bacteria and viruses inside the sterilizer chamber 101, significantly increasing the light exposure time. This optional mechanism can be made in many ways. In its simplest form, at least one positively charged plate inside the sterilizer chamber 101 can trap and collect the negatively charged particles on the air. Many bacteria and viruses are known to have a negative charged wall, so they can stick to a positively charged plate and their displacement gets slower. As shown in FIG. 3A, the plate 109 is pre-charged with positive charges. It is also practical to actively charge the plate 109 using an electrical charge generator 110 as illustrated on FIG. 3B. The electrical charge generator 110 can generate charge in many ways including but not limited to mechanically such as rubbing the materials, electrically such as using a high voltage, or electro-chemically. The electrical charge generator 110 can be placed inside the sterilizer chamber 101 or outside of the sterilizer chamber 101. In the case of the bacteria and viruses with positive charged wall, this invention is also practical using a negatively charged plate 109.

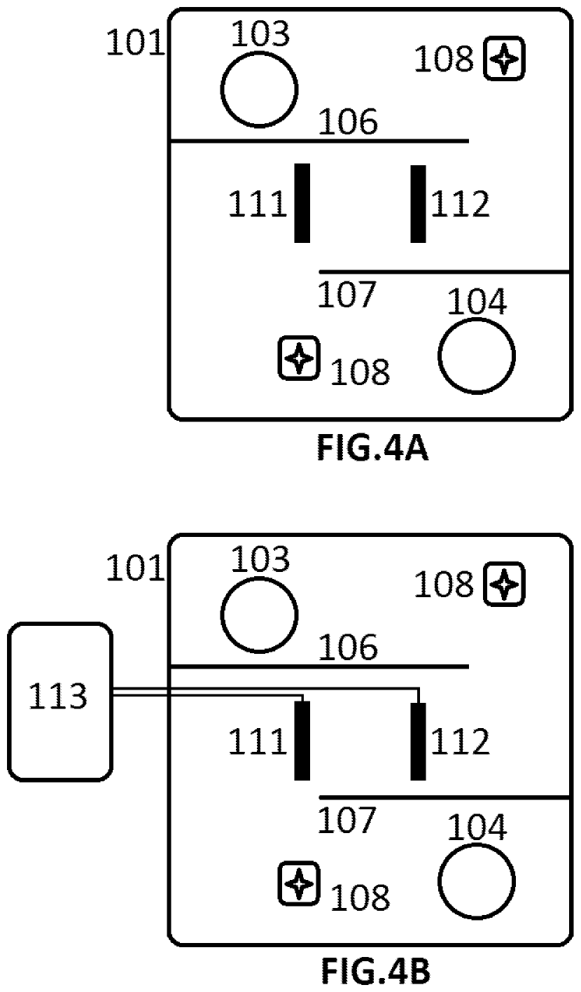

The electrostatic trap mechanism can also be made using one or multiple pair(s) of plates oppositely charged as shown in FIG. 4A and FIG. 4B. On its simplest form plate 111 is positively charged and the plate 112 is negatively charged, or the opposite: plate 112 is positively charged and the plate 111 is negatively charged, making an electric field between the two plates. The direction of this electric field is selected in a way that slows down the movement of the charged particles. It is also practical to actively charge the plate 111 and plate 112 using an electrical charge generator 113. The electrical charge generator 113 can generate charge in many ways including but not limited to mechanically such as rubbing the materials, electrically such as using a high voltage, or electro-chemically. The electrical charge generator 113 can be placed inside the sterilizer chamber 101 or outside of the sterilizer chamber 101.

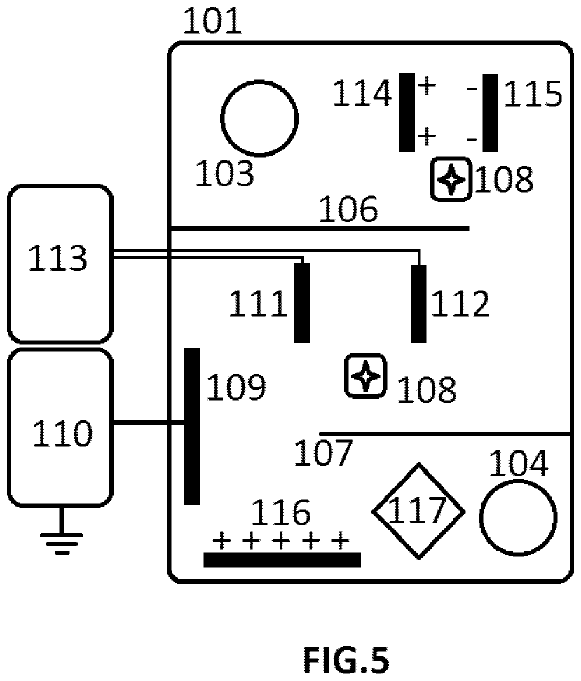

The electrostatic trap mechanism can also be made using any combination of one or multiple elements explained above. FIG. 5 is an example of the system comprising a pre-charged plate 116, the plate 109 charged with the electrical charge generator 113, a pair of pre-charged plate 114/plate 116, and a pair of plate 111/plate 112 charged with the electrical charge generator 110.

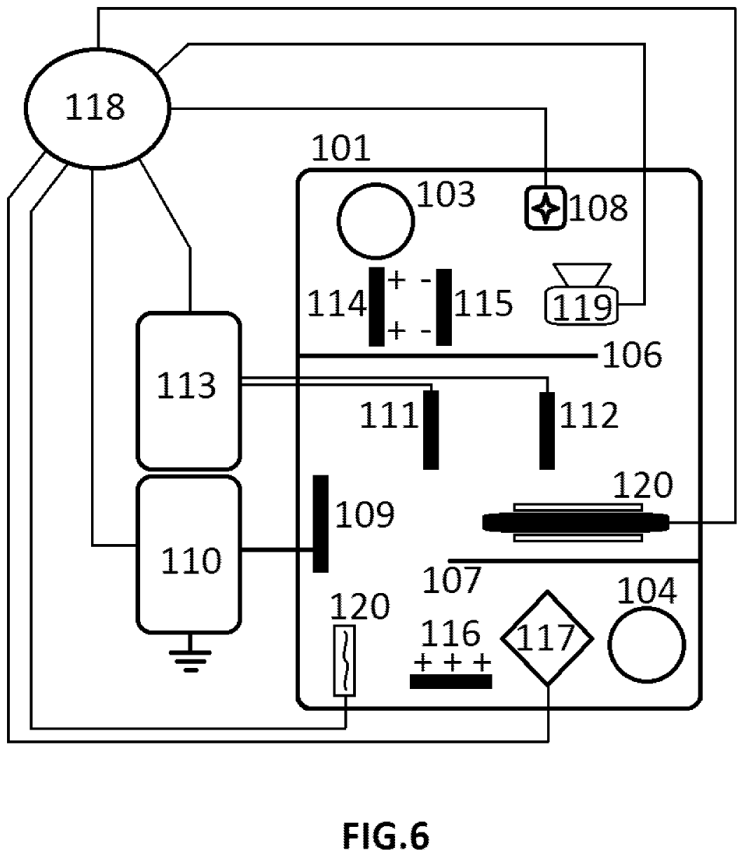

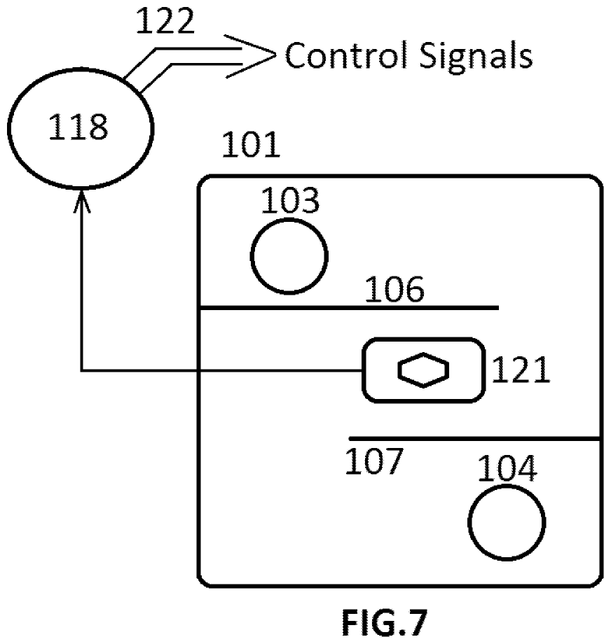

It must be noted that an air ionizer 117 can also pre-charge the air and particles on the air as shown on FIG. 5 to boost the effectiveness of the electrostatic trap mechanism. One or more air ionizer 117 can be added to any configurations as shown in FIG. 2, FIG. 3A, FIG. 3B, FIG. 4A, FIG. 4B, FIG. 5, FIG. 6, and FIG. 7.

The plate 109, plate 111, plate 112, plate 114, plate 115, and plate 116 can be placed anywhere and with any orientations inside the sterilizer chamber 101. However, it is more effective if they are placed in positions and with orientations that absorbs more light beams. These plates can be made in any shape and dimensions. They can also have a grid or mesh structure to let the air passing through them for installing these plates on the air flow path 105.

In this sterilizer face mask system the optical source 108 can be replaced with one or multiple devices that can kill, deactivate, or damage the microbial diseases, bacteria and viruses, it includes but is not limited to: sonic or ultrasonic device, heating element, cooling element, vibration element, and ionizer element. The sterilizer chamber 101 can also include any combination of these devices. For example a custom designed sterilizer face mask system can include 5 UV-C LEDS, one heating element, two ultrasonic transmitters, and one ionizer element in combination with the optional electrostatic trap mechanism.

This sterilizer face mask system may be equipped with at least one controller unit 118 that can turn on and off the bacteria killing/deactivating units and the electrical charge generator units and or adjust/control their intensity, energy, power, or effectiveness as shown in FIG. 6. The controller unit 118 can also be programmable by the manufacturer or programmable by the user. The controller unit 118 can be a microcontroller or a micro processor with supporting components. In an example embodiment, the controller unit 118 can control the electrical charge generator 110 and electrical charge generator 113, light source 108, sonic or ultrasonic transducer 119, heating/cooling element 120, and vibration generator 120. The controller unit 118 can be placed anywhere inside or outside of the sterilizer chamber 101.

Another novelty of this invention is the integration of a flow switch or a flowmeter to improve the power efficiency and thermal management of the system. As shown in FIG. 7, a flowmeter 121 is placed on the air flow path 105 to detect the existence of the air flow and or to measure the air flow rate and or to measure the speed of the air and or to measure the air flow direction. The data measured by flowmeter 121 is used by the controller unit 118 to activate or deactivate different units via control signal lines 122. The decision process to choose what component should be powered at what rate at what flow rate or flow direction can be pre-programmed at the manufacturing stage or be programmed by the user. For example using the control panel of the sterilizer face mask system, the user can program the instrument to power on the light sources only when there is an air flow from the window 103 to the window 104 and power off the light sources when there is no air flow inside the sterilizer chamber 101 and to power off the light sources when there is an air flow from window 104 to window 103. The user can also program the instrument to control the intensity of the light sources proportional to the air flow speed or the air flow rate. The similar programming can be done on other elements including: the electrical charge generator 110 and electrical charge generator 113, light source 108, sonic or ultrasonic transducer 119, heating/cooling element 120, and vibration generator 120.

For an example the controller unit 118 can control the electrical charge generator 110 to charge the plate 111 with positive charge and charge the plate 112 with the negative charge when there is an air flow from the window 103 to the window 104 and to charge the plate 112 with positive charge and charge the plate 111 with the negative charge when there is an air flow from the window 104 to the window 103. The amount of the charges can also be proportional with the air flow speed or the air flow rate.

With the similar logic the on/off cycle and the intensity/energy/power/effectiveness of the thermal (heating/cooling) element, vibration element, and the ultrasonic transducer can also be controlled based on the feedback from the flowmeter.

This ability will available amazing applications for this invention. For example, if the sterilizer face mask system is going to be used by a physician treating patients with the virus disease and having weakened immune system, the system should prevent the transfer of viruses from outside ambient to the user and prevent the spread of bacteria from the user to the ambient. In this application, the sterilizer face mask system can be programmed to only activate the elements that are effective on that specific virus at the inhalation phase of breathing and also to only activate the elements that are effective on the bacteria at the exhalation phase of breathing.

The flowmeter 121 can be any type of flowmeter including but not limited to: mechanical, optical, magnetic, or ultrasonic. In the case of the ultrasonic flowmeter, the flowmeter 121 can be implemented as a stand-alone flow meter or it can be integrated/merged with the ultrasonic transducer 119. The second case means that the ultrasonic transducer(s) of the flowmeter 121, while working at transmitting mode, their propagated ultrasonic wave will be employed to affect (kill or inactivate) the microbes, bacteria, or viruses.

1. An air sterilizer system comprising of a parasite killer unit and a one-way valve, wherein the said parasite killer unit is a light source, a heat source, a sonic source, an ultrasonic source, a cooling element, a vibration element, or a first ionizer, wherein the said parasite killer unit kills, weakens, or deactivates a parasite in an air inside the said system, wherein the said one-way valve forces an air inside the said system to flow in one direction.

2. The system of claim 1, wherein the said system is a face mask or the said system is a device attached to a face mask.

3. The system of claim 1, wherein the total volume of all synchronized sterilizer chamber of the said sterilizer is between 10% to 100% or equal to or larger than the tidal volume of a user of the said system.

4. The system of claim 1 further comprising of an air flow meter, wherein the said flow meter measures a flow rate or a flow direction or the existence of a flow of an air inside the said system, wherein the said system switches the said killer unit based on a data from the said air flow meter or the said system adjusts the intensity of an applied power by the said killer unit based on a data from the said air flow meter.

5. The system of claim 4 further comprising of a fan or an air pump, wherein the said system switches the said fan or the said air pump based on a data from the said air flow meter or the said system adjusts the speed or the power of the said fan or the said air pump based on a data from the said air flow meter.

6. The system of claim 1 further comprising of a parasite trap unit, wherein the said parasite trap unit comprises of an electrostatically charged object or an electrically charged plate charged by a power supply or two deferentially charged plates charged by a power supply, wherein the said parasite trap unit traps a parasite in an air or slows the motion of a parasite in an air inside the said system.

7. The system of claim 6 further comprising of an air flow meter, wherein the said flow meter measures a flow rate or a flow direction or the existence of a flow of an air inside the said system, wherein the said system switches the said power supply based on a data from the said air flow meter or the said system adjusts the applied power by the said power supply unit based on a data from the said air flow meter.

8. The system of claim 6 further comprising of a second ionizer, wherein the said second ionizer ionizes an air inside the said system.

9. An air sterilizer system comprising of a parasite killer unit and an air flow meter, wherein the said parasite killer unit is a light source, a heat source, a sonic source, an ultrasonic source, a cooling element, a vibration element, or a first ionizer, wherein the said parasite killer unit kills, weakens, or deactivates a parasite in an air inside the said system, wherein the said flow meter measures a flow rate or a flow direction or the existence of a flow of an air inside the said system, wherein the said system switches the said killer unit based on a data from the said air flow meter or the said system adjusts the intensity of an applied power by the said killer unit based on a data from the said air flow meter.

10. The system of claim 9, wherein the said system is a face mask or the said system is a device attached to a face mask.

11. The system of claim 9, wherein the total volume of all synchronized sterilizer chamber of the said sterilizer is between 10% to 100% or equal to or larger than the tidal volume of a user of the said system.

12. The system of claim 9 further comprising of a parasite trap unit, wherein the said parasite trap unit comprises of an electrostatically charged object or an electrically charged plate charged by a power supply or two deferentially charged plates charged by a power supply, wherein the said parasite trap unit traps a parasite in an air or slows the motion of a parasite in an air inside the said system.

13. The system of claim 12 further comprising of a fan or an air pump, wherein the said system switches the said fan or the said air pump based on a data from the said air flow meter or the said system adjusts the speed or the power of the said fan or the said air pump based on a data from the said air flow meter.

14. The system of claim 12 further comprising of a second ionizer, wherein the said second ionizer ionizes an air inside the said system.

15. The system of claim 14 further comprising of a fan or an air pump, wherein the said system switches the said fan or the said air pump based on a data from the said air flow meter or the said system adjusts the speed or the power of the said fan or the said air pump based on a data from the said air flow meter.

16. The system of claim 9 further comprising of a fan or an air pump, wherein the said system switches the said fan or the said air pump based on a data from the said air flow meter or the said system adjusts the speed or the power of the said fan or the said air pump based on a data from the said air flow meter.

17. An air filtering system comprising of a parasite trap unit and an air flow meter, wherein the said parasite trap unit comprises of an electrically charged plate charged by a power supply or two deferentially charged plates charged by a power supply, wherein the said parasite trap unit traps a parasite in an air or slows the motion of a parasite in an air inside the said system, wherein the said flow meter measures a flow rate or a flow direction or the existence of a flow of an air inside the said system, wherein the said system switches the said parasite trap unit based on a data from the said air flow meter or the said system adjusts the intensity of the said parasite trap unit based on a data from the said air flow meter.

18. The system of claim 17, wherein the said system is a face mask or the said system is a device attached to a face mask.

19. The system of claim 17 further comprising of a second ionizer, wherein the said second ionizer ionizes an air inside the said system.

20. The system of claim 19 further comprising of a fan or an air pump, wherein the said system switches the said fan or the said air pump based on a data from the said air flow meter or the said system adjusts the speed or the power of the said fan or the said air pump based on a data from the said air flow meter.

21. The system of claim 17 further comprising of a fan or an air pump, wherein the said system switches the said fan or the said air pump based on a data from the said air flow meter or the said system adjusts the speed or the power of the said fan or the said air pump based on a data from the said air flow meter.
